(19) United States
(12) Patent Application Publication
(10) Pub. No.: US 2019/0320934 A1
(43) Pub. Date: Oct. 24, 2019

(54) MEDICAL IMAGE ACQUISITION WITH SEQUENCE PREDICTION USING DEEP LEARNING

(71) Applicant: Siemens Healthcare GmbH, Erlangen (DE)

(72) Inventors: Benjamin L. Odry, West New York, NJ (US); Boris Mailhe, Plainsboro, NJ (US); Mariappan S. Nadar, Plainsboro, NJ (US)

(21) Appl. No.: 16/280,349

(22) Filed: Feb. 20, 2019

Related U.S. Application Data
(60) Provisional application No. 62/659,251, filed on Apr. 18, 2018.

(51) Int. Cl. A61B 5/055; A61B 5/00; G06T 7/00; G01R 33/54

(52) U.S. Cl. CPC A61B 5/055; A61B 5/7264; G16H 30/40; G01R 33/54; G06T 7/0012

(57) ABSTRACT

Automated sequence prediction is provided for a medical imaging session including a self-assessment mechanism. An initial scout sequence is performed of a patient or object. The initial scout sequence is validated. An abbreviated acquisition protocol is performed. The abbreviated acquisition protocol is validated. Additional sequences are performed. The sequences may also be configured based on the analysis of the previous scans using deep learning-based reasoning to select the next appropriate settings and procedures.

### CROSS REFERENCE TO RELATED APPLICATIONS

This application claims the benefit of U.S. Provisional Application No. 62/659,251 filed on Apr. 18, 2018 which is hereby incorporated by reference in its entirety.

### FIELD

The present embodiments relate to medical image acquisition and processing.

### BACKGROUND

Diagnostic imaging procedures may include multiple different procedures or tasks. In an example, magnetic resonance imaging includes a multitude of different possible scans. The range and complexity of magnetic resonance imaging sequences and procedures available presents a clinician with difficult choices. Each sequence includes its own physics, characteristics, and output. Each sequence may provide different results for that are useful for different medical applications. In addition, a clinician may not be able to predict the results of a single scan thus requiring multiple different procedures. When performing a medical imaging procedure, an operator must determine the appropriate scan for the patient while the patient is situated and prepped. Any delays or omissions may lead to inefficiencies or slower diagnostic outcomes. The complexity of imaging and the inability to predict the needs prior to the scan present a problem for medical imaging acquisitions.

### SUMMARY

By way of introduction, the preferred embodiments described below include methods and systems for automated acquisition with sequence prediction. An initial scout sequence is performed to provide guidance for future scans. An abbreviated acquisition protocol is then performed that identifies abnormalities and provides direction as to future sequences.

In a first aspect, a method is provided for automated image acquisition of a patient using a magnetic resonance imaging system. First MR data is acquired by the magnetic resonance imaging system using a first imaging protocol. The first MR data is validated. A second imaging protocol is ordered. Second MR data is acquired using the second imaging protocol. The second MR data is validated. A third imaging protocol is ordered until the desired number of contrasts is reached, to the satisfaction of the clinician.

In a second aspect, a method is provided for automated medical image acquisition of a patient. A magnetic resonance imaging system acquires first MR data using a scout acquisition sequence. Landmarks are detected in the first MR data. Abnormal regions can be identified in the first MR data. An abbreviated acquisition protocol is determined depending on the identified landmarks and whether or not abnormal regions are identified Second MR data is acquired using the abbreviated acquisition protocol. Anomalies are identified in the second MR acquired sequences. One or more additional acquisition sequences are determined as a function of the identified anomalies. The one or more additional acquisition sequences are performed.

In a third aspect, a system is provided for automated medical image acquisition of a patient. The system includes a magnetic resonance imaging device, a memory, and a control unit. The magnetic resonance imaging device is configured to acquire first MR data using a first imaging protocol and second MR data using a second imaging protocol. The memory is configured to store the first MR data and second MR data. The control unit is configured to validate the first MR data, order the second imaging protocol as a function of the validation of the first MR data, validate the second MR data, and order a third imaging protocol as a function of the validation of the first MR data and second MR data.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 depicts an example MR system.

FIG. 2 depicts an example method for automated acquisition of medical imaging data.

FIG. 3 depicts an example method for automated acquisition for providing guidance in a medical imaging procedure.

FIG. 4 depicts an example method for detecting abnormal regions in medical imaging data.

FIG. 5 depicts an example method for classifying abnormalities in medical imaging data.

FIG. 6 depicts an example method for identifying additional sequences in a medical imaging procedure.

FIG. 7 depicts an example system for automated acquisition of medical imaging data.

### DETAILED DESCRIPTION

Automated sequence prediction is provided for a medical imaging session including a self-assessment mechanism. An initial scout sequence is performed of a patient. The initial scout sequence is validated. An abbreviated acquisition protocol is performed. The abbreviated acquisition protocol is validated. Additional sequences are performed. In an embodiment, subsequent scanning sequences may be configured based on the analysis of the previous scans using deep learning-based reasoning to select the next appropriate settings and procedures.

Medical imaging refers to several different technologies that are used to view the human body in order to diagnose, monitor, or treat medical conditions. Each type of technology gives different information about the area of the body being studied or treated, related to possible disease, injury, or the effectiveness of medical treatment. When performing a medical imaging procedure on an object or patient, multiple scans may be performed in order to capture the needed information to make an accurate diagnosis or identify a proper treatment. Determining which scans to perform during a procedure is a complex task that includes many inefficiencies.

Different methods have been used to overcome the complexity and issues. A clinician may view the results as the results are generated and make a decision of whether or not to proceed. Alternatively, a multitude of scans may be ordered to not miss any possible scans in order to save the patient a trip back to the hospital at a later date. Each of the methods may result in under or over scanning a patient. For the manual process, an attending physician may be required at all times in order to manually review results. Further, manually determining the sequences to be performed may be error prone, particularly under the timing pressure of requiring a decision while a patient is still in the hospital or scanning machine. Ordering a full slate of sequences is inefficient.

Embodiments provide systems and methods for automating the acquisition of imaging data of a patient or object. An initial scan is used to scout the patient or object and determine settings for subsequent sequences. The result of each scan may be validated prior to performing additional scans. The output of each scan may be processed by different machine trained networks to identify abnormalities or abnormal regions. The identified abnormalities or abnormal regions may be automatically analyzed to determine subsequent scans and procedures.

In the embodiments described below, the imaging system is a magnetic resonance (MR) imaging system. Other imaging systems may be used such as computed tomography (CT) or ultrasound. In addition, the examples below describe the embodiment using a scan of a patient's brain. Other regions or organs of a patient may be scanned, for example, the lungs, breast, or heart.

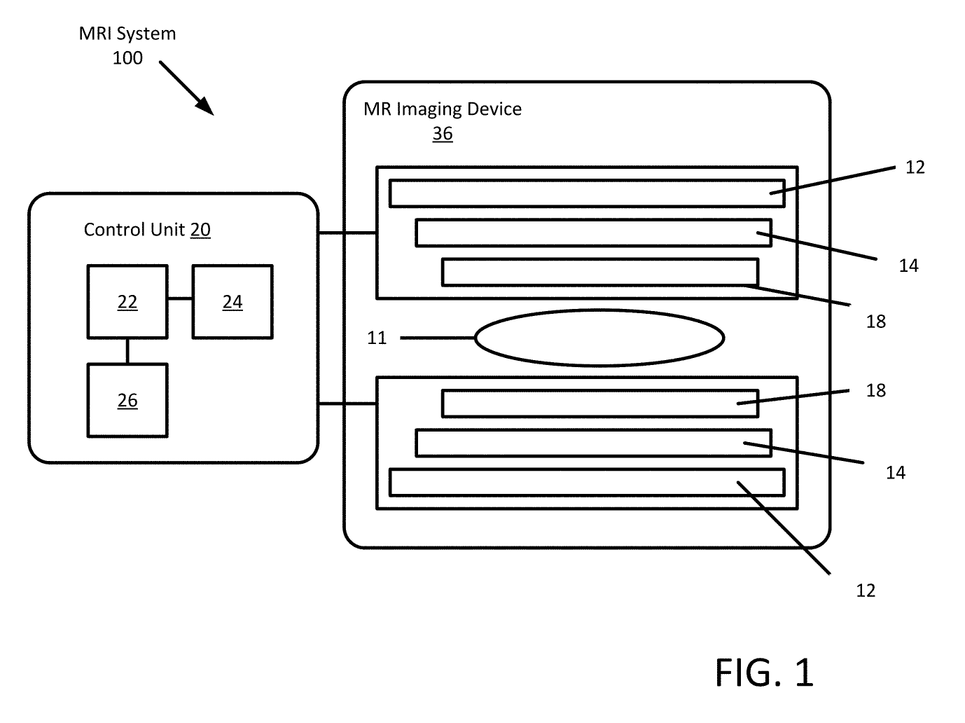

FIG. 1 depicts an MR system 100 for acquisition of frequency domain components representing MR data for storage in a storage array. The MR system 100 includes a control unit 20 configured to process the MR signals and generate images of the body for display to an operator. The control unit 20 may store the MR signals and images in a memory 24 for later processing or viewing. The control unit 20 may include a display 26 for presentation of images to an operator. The MR scanning system 100 is only exemplary, and a variety of MR scanning systems may be used to collect the MR data.

In the MR system 100, magnetic coils 12 create a static base or main magnetic field B₀ in the body of patient 11 or an object positioned on a table and imaged. Within the magnet system are gradient coils 14 for producing position dependent magnetic field gradients superimposed on the static magnetic field. Gradient coils 14, in response to gradient signals supplied thereto by a gradient and control unit 20, produce position dependent and shimmed magnetic field gradients in three orthogonal directions and generate magnetic field pulse sequences. The shimmed gradients compensate for inhomogeneity and variability in an MR imaging device magnetic field resulting from patient anatomical variation and other sources.

The control unit 20 may include a RF (radio frequency) module that provides RF pulse signals to RF coil 18. The RF coil 18 produces magnetic field pulses that rotate the spins of the protons in the imaged body of the patient 11 by ninety degrees or by one hundred and eighty degrees for so-called “spin echo” imaging, or by angles less than or equal to 90 degrees for “gradient echo” imaging. Gradient and shim coil control modules in conjunction with RF module, as directed by control unit 20, control slice-selection, phase-encoding, readout gradient magnetic fields, radio frequency transmission, and magnetic resonance signal detection, to acquire magnetic resonance signals representing planar slices of the patient 11.

In response to applied RF pulse signals, the RF coil 18 receives MR signals, e.g. signals from the excited protons within the body as the protons return to an equilibrium position established by the static and gradient magnetic fields. The MR signals are detected and processed by a detector within RF module and the control unit 20 to provide an MR dataset to a processor 22 for processing into an image. In some embodiments, the processor 22 is located in the control unit 20, in other embodiments, the processor 22 is located remotely. A two or three-dimensional k-space storage array of individual data elements in a memory 24 of the control unit 20 stores corresponding individual frequency components including an MR dataset. The k-space array of individual data elements includes a designated center, and individual data elements individually include a radius to the designated center.

A magnetic field generator (including coils 12, 14 and 18) generates a magnetic field for use in acquiring multiple individual frequency components corresponding to individual data elements in the storage array. The individual frequency components are successively acquired using a Cartesian or other spatial acquisition strategy as the multiple individual frequency components are sequentially acquired during acquisition of an MR dataset. A storage processor in the control unit 20 stores individual frequency components acquired using the magnetic field in corresponding individual data elements in the array. The row and/or column of corresponding individual data elements alternately increases and decreases as multiple sequential individual frequency components are acquired. The magnetic field generator acquires individual frequency components in an order corresponding to a sequence of substantially adjacent individual data elements in the array, and magnetic field gradient change between successively acquired frequency components is substantially minimized.

One use of MR imaging is in studying a patient's brain. When studying a patient's brain, different MRI scans may be performed using different protocols and sequences. An MRI sequence is a specific setting of pulse sequences and pulsed field gradients, resulting in a particular image appearance. A multiparametric MRI is a combination of two or more sequences. Examples of MRI sequences include T1 weighted sequences, contrast sequences, fat suppression, T2 weighted sequences, fluid attenuated (FLAIR), diffusion weighted, perfusion weighted, among others. Each sequence may also include different variations for parameters or settings. An MRI protocol is a combination of various MRI sequences, configured to assess a particular region of a body of the patient and or pathological process. In an example, one standardized brain MRI protocol includes 3D T1-weighted, 3D T2-FLAIR, 3D T2-weighted, post single-dose gadolinium-enhanced T1-weighted, and a diffusion-weighted sequence. An MRI procedure includes one or more MRI protocols or MRI sequences that are performed during an imaging session. As an example, in an embodiment described below, an MRI procedure may include a scout acquisition sequence, an abbreviated acquisition protocol (that includes one or more sequences), and one or more additional sequences.

MRI protocols and procedures may be specific to a hospital or center where the imaging session occurs. Different physicians may order different sequences to be performed under different protocols or procedures. In many cases, standardized protocols may be used to simply the process. The use of standardized protocols, however, leads to inefficiencies. Additional scans are performed when not needed (and not used in the eventual diagnosis or treatment). Scans that might be useful may not be performed, leading to a delayed diagnosis or treatment.

Embodiments provide an intelligent way to scan a patient. A first scout sequence is used to “scout” the patient. A subsequent abbreviated imaging protocol is then performed based in part on the findings of the scout sequence. The results of the abbreviated imaging protocol may be analyzed by a network or model trained using deep learning techniques to identify which if any subsequent sequences should be performed. The abbreviated acquisition protocol may vary depending on the organ that is imaged and the results of the scout sequence. In case of neurological exam, a scout sequence is done first to determine organ localization. Then, structural (T1w, T2w, FLAIR) and diffusional (ADC, TraceW) scans may be performed as part of an abbreviated acquisition protocol. Depending on the medical institution, other scans such as SWI or GRE may also be part of the abbreviated acquisition protocol. The abbreviated acquisition protocol encapsulates the minimum number of contrasts needed to detect abnormality in the brain, breast, or other organs. For further characterization, additional scans (contrasts/sequences) may be prescribed. Embodiments may select or provide an offer to select the scans based on the set of findings from the abbreviated protocol. For example, in the case of specific pathologies (Brain tumor), additional acquisitions, such as Perfusion, T1w with Contrast and more are needed and may be ordered based on the results of the abbreviated acquisition protocol. Embodiments provide a way to automate the workflow after the abbreviated protocol is done and then, from the sequences or contrasts, use a network trained using deep learning to decide the appropriate next sequence(s). In addition, scout images may also give an idea of the presence or not of abnormalities in the organs and therefore, may act as a pre-abbreviated protocol and already give insight for the deep learning network to select the set of sequences to do next.

The disclosed embodiments may be implemented to computationally facilitate processing of medical imaging data and consequently improving and optimizing medical diagnostics. By using an automated workflow, errors in the scanning process are diminished and outcomes are improved. The use of an automated acquisition procedure is efficient in that a correct number of resources are used to acquire the needed medical images for diagnosis. The use of an automated acquisition procedure further limits errors by removing user errors and decisions from the process. The automated acquisition process not only automates image acquisition in general but also automatically tailors the process for each patient. The generated patient-specific process saves time for both the patient and any personal that reviews the images.

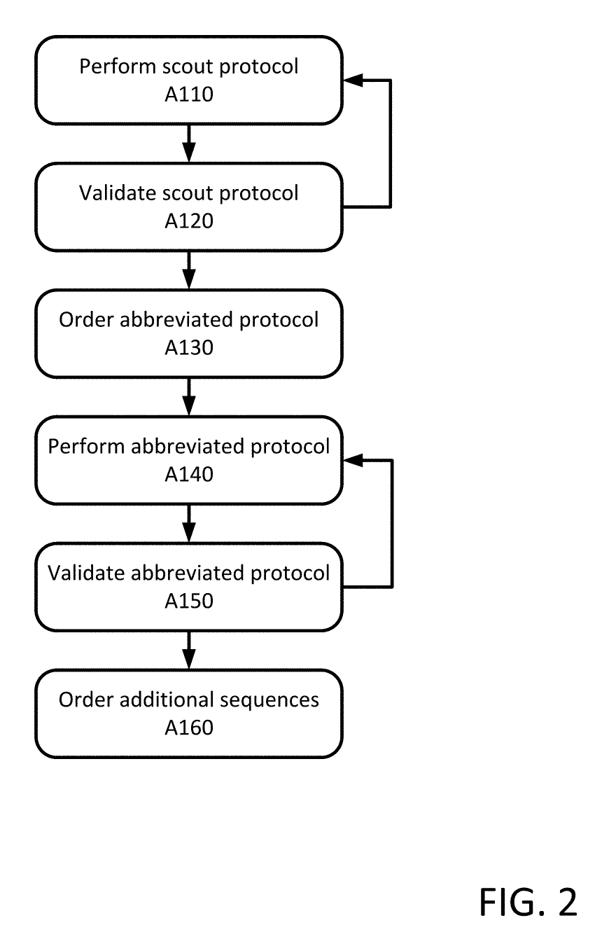

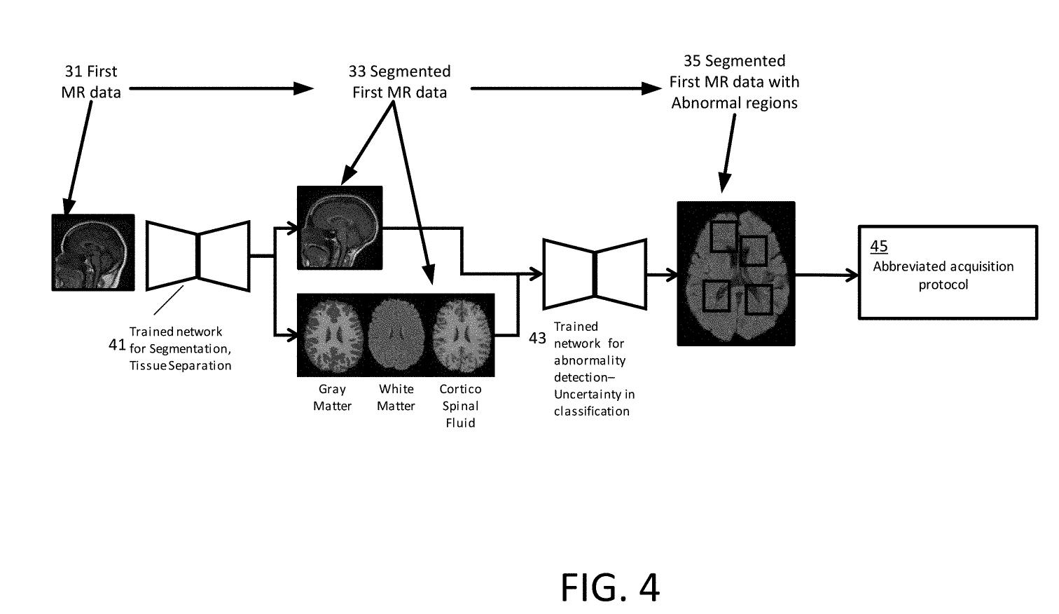

FIG. 2 depicts an example flowchart for providing automated image acquisition for a magnetic resonance imaging system. During application, the method provides a tailored image acquisition for the patient undergoing an imaging procedure. As a result, the certain acts may be omitted or changed depending on the results of the previous acts and the status of the patient. In an example, the acts may be paused, repeated, skipped, or stopped after each scan if the acquisition is not validated or as a result of an analysis of the results. The acts are performed by the system of FIG. 1, FIGS. 4-7, other systems, a workstation, a computer, and/or a server. Additional, different, or fewer acts may be provided. The acts are performed in the order shown (e.g., top to bottom) or other orders.

The validation and analysis of the acquired MR data may be performed by networks or models trained using machine learning techniques. The networks or models may be trained prior to the act of FIG. 2. Training data may be acquired and used to configure the network or models. The networks or models may be updated as new training data is acquired or changes are made to the system. In an example, the machine trained agent of act A120 is applied at act A120 but may be trained at a prior point in time using machine learning techniques.

At act A110, first MR data is acquired using a first imaging protocol. The first protocol may include at least a medium or low-resolution sequence. In an embodiment, multiple sequences may be performed to acquire the first MR data. The first MR data may be acquired directly using an MRI system. As depicted and described in FIG. 1 above, MR data may be acquired using MR scanners. For example, gradient coils, a whole-body coil, and/or local coils generate a pulse or scan sequence in a magnetic field created by a main magnet or coil. The whole-body coil or local coils receive signals responsive to the re-orientation of molecules shifted due to the scan sequence. In an embodiment and used as an example below, MR data may represent image data for a brain of a patient. Different objects, organs, or regions of a patient may also be scanned.

MR data may be k-space data or image data. Image data may be MR data after Fourier transform into object space. The image data may be at any point after transform, so may be scalar values or may be formatted as RGB values for a display screen. MR data or image data may be scan data to be used to generate an image on a display. MR data may be data being processed to generate an image, data formatted for display, or data that has been used to display. MR data may be data with no or some image processing.

In an embodiment, the MR data may represent a volume. Three-dimensional datasets are obtained. As k-space data, information content may be provided that is responsive to a three-dimensional distribution of locations, but the data itself does not directly represent the locations prior to transform. In alternative embodiments, two-dimensional datasets representing or responsive to tissue in planes are obtained. In other embodiments, sequences of MR data responsive to the same tissue over time are acquired for training.

Alternative methods may be used to acquire the MR data. The MR data may be acquired remotely from the server or workstation. The MR data may be stored locally onsite or offsite, for example in the cloud.

As used herein, MR data includes both raw MR data and processed MR data. Processed MR data may include image and volume data. MR data may include 2D images, sequences of 2D images, 3D volumetric imagery, or sequence of 3D volumetric imagery. If the MR data is defined in 3D space (e.g., obtained from a series of MR images), each image “slice” may be provided individually in a “slice-by-slice” manner. Alternatively, the MR data may be acquired as 3D volumetric data directly. The examples described herein use three-dimensional MR data referred to as volumes. Additionally, the terms MR data and volume may be used interchangeably in that the MR data represents at least one volume. Volumes are encoded using an array of elements referred to as voxels. A voxel represents a value on a regular or irregular grid in three-dimensional space. Two-dimensional MR data may be encoded using a bitmap of pixels.

At act A120, the first MR data is validated. The validation may be based on an assessment mechanism of landmark detection or assessment of a registration. For the landmark detection, one or more landmarks are identified in the first MR data. The first MR data may be used to define acquisition matrices along specific anatomical regions. The detected landmarks may be used to define the acquisition matrices by identifying landmarks in the first MR data. The acquisition matrix defines the area or region to be scanning by defining a number of independent data samples that are acquired by the imaging scan in the frequency (f) and phase (f) directions.

Deep learning techniques are conventionally applied to various problems ranging from image classification, object detection and segmentation. Deep learning is the automatic learning of hierarchical data representations describing the underlying phenomenon. That is, deep learning proposes an automated feature design by extracting and disentangling data-describing attributes directly from the raw input in contrast to feature handcrafting. Hierarchical structures encoded by neural networks may be used to model the learning approach.

Deep reinforcement learning (DRL) is a type of deep learning that uses a machine trained agent. The machine trained agent is generated and trained to self-develop an optimized method for efficiently identifying an anatomical landmark. For landmark detection, the agent learns (e.g., develops a landmark detection solution) during training with a set of training images that each contains annotated landmarks. Focusing on one particular landmark indexed in each training example, the DRL method trains an artificial, intelligent agent that automatically discover strategies for finding the chosen landmark not only in the provided data, but also in unseen examples.

Large numbers of search parameters evolve over the course of training the agent on a set of identified landmark targets. The agent inputs a training set, randomly navigating through the image via the state space. Gradually, the agent learns a policy during training to optimize the expected reward value r(t) of its actions. Expected rewards are determined by the reward value of the possible actions, a, available to the agent at time, t with the goal of identifying the target landmark (via maximizing expected reward value). Actions define the positional movement that occurs during state space transitions with respect to the state space's proximity to the target landmark. Sequential actions are determined and stored by the agent, and simultaneously with landmark detection, eliminating the need to hand-craft optimization criteria, image features, or exhaustive image search. The artificial agent may be applied to object detection, segmentation, tracking, and/or image registration. In order to learn optimal action policy in a sequence of learning episodes, the agent is given random training images with corresponding random start-states. The agent then follows an E-greedy search strategy in the selected image, generating, at the end of the episode a trajectory which is added to its experience memory. During the exploration, periodic updates are applied to the parameters of the neural network, leading to a more accurate approximation of the optimal Q* function, given the current experience. This process is repeated in an iterative manner until the detection accuracy on the validation set is minimal.

Once trained, the machine trained agent is able to identify landmarks in the acquired first MR data. The landmarks may be used for registration, setting the acquisition matrix, and validating the first MR data. The MR data may also be registered, using, for example, an AutoAlign algorithm. AutoAlign provides an output registration matrix that may be utilized to align the MR data to an identified and consistent anatomic orientation. The identified landmarks and registration may be used to both validate the first MR data and provide guidance for future sequences.

The agent may also provide a confidence value for the landmark detection that represents the confidence of the agent in the landmark detection. The registration may also be assessed to determine if the MR data is consistent. The AutoAlign algorithm includes a feedback mechanism that measures and reports alignments which have the potential to be outside of stated specifications defined by an atlas. The feedback mechanism using a “Measurement Index” value that is the average of a distance between a point P and a distribution D for the intensity of all atlas points to the patient images supplied for alignment. The higher the value of the measurement index, the lower of the probability of an alignment within stated specifications between the atlas and the patient acquired MR data, although not all alignments with a relatively high measurement index value indicate a poor alignment. Any differences between acquired MR data and the normalized atlas will generate some positive level of Measurement Index value, however, this may not indicate an error. For validation, the measurement index value of the MR data may be compared against a threshold value.

Based on the confidence value and or measurement index value, the system may proceed to the abbreviated acquisition protocol. If, for example, the score or measurement index value indicates that the MR data is not acceptable, the protocol may be repeated until the MR data is sufficiently accurate.

At act A130, a second imaging protocol is ordered at least in part as a function of the analysis of the first MR data performed during validation. The detected landmarks may also be used to define the acquisition matrix of the second imaging protocol. As landmarks correspond to specific anatomical regions in the brain, position of the head in the scan may be estimated, given that there are enough landmarks to characterize the position. As an example, a non-exhaustive subset of the landmarks may include “Crista Galli, Bregma, left right Orbit, Occipital Bone” that are spread out enough to map the position of the head. Additional vascular or tissue landmark may also be used, for example. The acquisition matrix defines the resolution for a sequence. Sequences in the second image protocol may be configured to provide an accurate and useful scan by following the guidance provided by the first MR data.

The second imaging protocol may be abbreviated and thus referred to as an abbreviated acquisition protocol or abbreviated acquisition protocol. The second imaging protocol is abbreviated in that the protocol does not include all possible sequences, but rather a select few that can be performed quickly and efficiently. Additional sequences may then be automatically ordered as a result of the findings of the abbreviated acquisition protocol.

In an example, the abbreviated acquisition protocol may include one or more sequences such as structural (T1w, T2w, FLAIR) or diffusional (ADC, TraceW) scans. SWI or GRE may also be part of the abbreviated acquisition protocol. In an embodiment, the abbreviated acquisition protocol include scanning at a higher resolution than the scout protocol (the first MR data).

The number and types of sequences in the abbreviated acquisition protocol may be influenced by the results of the first MR data. During registration or landmark detection of the first MR data, the system may identify one or more abnormalities or issues. Subsequent scans including scans in the abbreviated acquisition protocol may be generated light of the abnormalities or issues. The abnormalities may include abnormal structures of the organ or surrounding tissues. The focus or region of the sequences of the abbreviated acquisition protocol may be influenced by the registration and landmark detection. As each patient is different, each scan may be tailored to the patient based on the findings of the scout sequence. Two patients that start with the same scout acquisition sequence may undergo different abbreviated acquisition protocols based on the findings of the scout acquisition sequence. Additional sequences and procedures may differ based on the findings of the abbreviated acquisition protocol.

At act A140, second MR data is acquired using the second imaging protocol. After acquisition, at act A150, the second MR data is validated. In an embodiment, each of the one or more sequences of the abbreviated acquisition protocol and the respective output are validated as the sequences are performed. Each sequence may be performed and then validated, or the entirety of the abbreviate protocol (including multiple sequences) may be performed and then validated. If the sequences are not validated, the system may not move on to the next sequence in the protocol. If a sequence fails validation, the sequence may be rerun, altered and rerun, or skipped.

Validation for the sequences of the abbreviated acquisition protocol may include determining an alignment or misalignment of the second MR data with respect to the matrices acquired from first MR data. For instance, when using a simultaneous multi-slice protocol, slices from the second MR data may be registered to the first MR data to ensure that misalignment is minimal. Additionally, or alternatively, the sequences of the abbreviated acquisition protocol may be validated by checking that the quality of the acquisition is correct, e.g. there is an acceptable amount of motion, bias field, noise. Any automated technique may be used to check for the amount of motion, bias field, or noise. Thresholds may be defined for each of motion, bias field, or noise. If the threshold is exceeded, the sequence may fail validation. If a sequence or set of acquired MR data fails validation, the sequence may be repeated, altered to fix any issues and repeated, or skipped. The process and alteration may be performed automatically, or the procedure may pause and require input from a clinician. As mentioned above, the abbreviated acquisition protocol may be stopped at any point after a sequence if the findings are not validated.

At act A160, additional sequences are automatically ordered as a function of the findings of the second image protocol. The additional sequences may be configured using the registration or landmarks identified in the output data from the scout and abbreviated acquisition protocols. The specific sequences that are ordered may be based in part on the finding of the scout and/or abbreviated acquisition protocols. For example, additional scans to the actual abbreviated acquisition protocol may be configured to target regions of the organ where there might be abnormality, e.g. to generate additional focused high-resolution scans on the suspicious regions. High resolution scans may be time and equipment intensive. By providing guidance from the scout and abbreviated acquisition protocols, the high-resolution additional sequences may be narrowly tailored to any issues that have been identified in the patient up to this point. Other scan sequences or protocols that may not provide a benefit given the findings may not be performed. Additionally, the process may stop at any point after analyzing the previous scan.

The additional sequences may be performed to acquire third MR data. The third MR data may be displayed or provided to a user. The first MR data and second MR data may also be displayed or provided to a user. The first, second, and third MR data may be stored for later analysis.

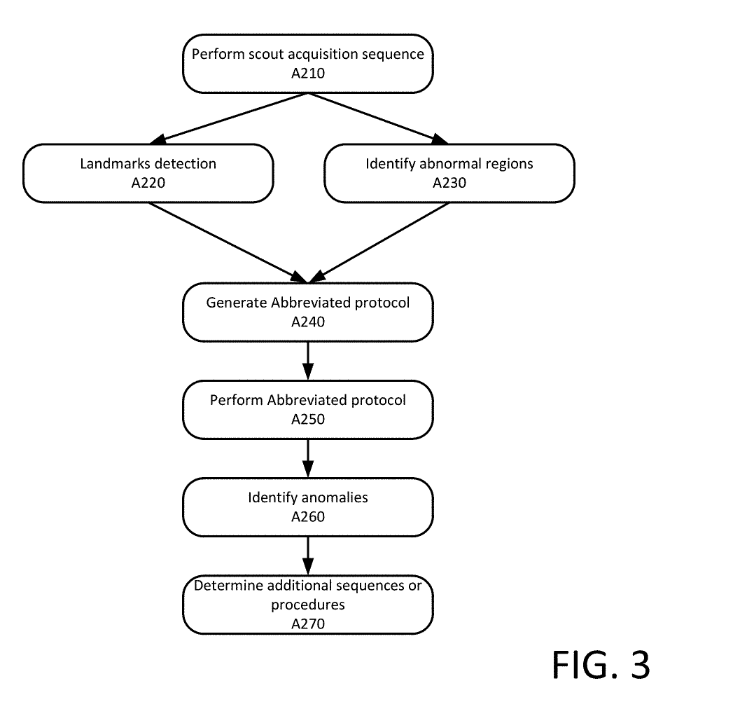

FIG. 3 depicts a workflow for automated image acquisition of a patient. The acts are performed by the system of FIG. 1, FIGS. 4-7, other systems, a workstation, a computer, and/or a server. Additional, different, or fewer acts may be provided. The acts are performed in the order shown (e.g., top to bottom) or other orders. During application, the method provides a tailored image acquisition for the patient undergoing an imaging procedure. As a result, the certain acts may be omitted or changed depending on the results of the previous acts and the status of the patient. In an example, different acquisition sequences may be ordered, paused, repeated, skipped, or stopped if the acquisition is not validated or as a result of an analysis of the results.

At act A210, first MR data is acquired using a first sequence. The first sequence may be a medium (for example, 2 mm isotropic), or low-resolution sequence. In an embodiment, multiple sequences may be performed to acquire the first MR data. The first MR data is acquired using a magnetic resonance imaging system. The first sequence may be referred to as a scout sequence or scout protocol.

At acts A220, landmarks, positioning, and/or coverage may be detected and identified in the first MR data. As described above, landmarks and positioning may be determined using registration or networks trained with deep learning techniques. In an embodiment, DRL may be used to train an agent to identify landmarks in the first MR data. DRL is a technique facilitating learning as an end-to-end cognitive process for an artificial agent, instead of a predefined methodology. The artificial agent interacts with an uncertain environment (e.g., medical image of a patient without landmark target identified) with the target of reaching pre-determined goals (e.g., identifying the landmark target in the image). The agent can observe the state of the environment and choose to act on the state, similar to a trial-and-error search, maximizing the future reward signal received as a response from the environment. In an example, for a scout brain MR data, the detected landmarks may include, for example, the Crista Galli, Bregma, Foragen Magnum, Orbits, Sella, Optical Nerves and Chiasm, among others. The detected landmark allow the imaging system to properly configure future sequences by positioning the acquisition matrix and also establishing deviation of the positions of the landmarks with respect to those of normal patients. Registration and positioning may also be calculated using an AutoAlign algorithm as described above.

At act A230, abnormal regions are identified in the first MR data. A global intensity distribution for a region of the scan may indicate whether there's a deviation from normal and where. The global intensity distribution may be analysis using one or more networks trained using deep learning techniques. FIG. 4 depicts an example flowchart for determining abnormal regions in the first MR Data 31. As depicted in FIG. 4, the first MR data 31 is segmented and classified by a first network 41 that is trained for segmentation and tissue separation.

The MR data may be segmented using any segmentation method. Segmentation is the process of dividing an input into different parts or sections, e.g. for medical imaging, delineating the boundaries, or contours, of various tissues or structures in the body. Segmentation may also include classification. Classification assigns a label to the MR data, e.g. normal or abnormal, level of severity, a diagnosis, or type of tissue. Classification may assign to each element in the image a tissue class when the classes are defined in advance. In the case of brain MR, for tissue classification, image elements may be classified into three main tissue types: white matter (WM), gray matter (GM), and cerebrospinal fluid (CSF). Classification of the tissue types requires segmentation of the MR data into different parts. Image segmentation can be performed on two dimensional images, sequences of two-dimensional images, three-dimensional volume, or sequences of three-dimensional volumes. If the data is defined in three-dimensional space (e.g., obtained from a series of MR images), each image slice may be segmented individually in a slice-by-slice manner. The two-dimensional slices are then connected into a 3D volume or a continuous surface.

In an embodiment, the network 41 (segmentation network) is trained using an adversarial process (e.g. using a generative adversarial network or GAN). The GAN includes a generator network and a discriminator network. During the training process, the generator network attempts to generate output that can fool the discriminator network into thinking that the output is from the training set of data. In the adversarial process, the generator network may be trained to minimize the sum of two losses: a supervised L1 distance of the generator prediction, and an unsupervised adversarial term. The adversarial term is provided by the discriminator network. While the generator network is being trained, the discriminator network is also adjusted to provide better feedback to the generator network.

The discriminator network may use probability distributions of the real images (ground truth/training data) and the segmented generator images to classify and distinguish between the two types of images. The discriminator network provides the information to the generator network. The information provided by the discriminator network may be in the form of a gradient that is calculated as a function of a comparison of the probability distributions of the images, e.g. comparing a first probability distribution of values for the generated image with an expected probability distribution of values for the ground truth image. The gradient may include both a direction and a slope that steer updates for the generator network in the right direction. After a number of iterations, the gradient directs the generator network to a stable place where the generator network is generating images with probability distributions that are similar to the ground truth images. The gradients provided by the discriminator network change as the generator network generates and provides new images.

The training data for the GAN (and other networks) may include ground truth data or gold standard data. Ground truth data and gold standard data is data that includes correct or reasonably accurate labels. For the segmentation problem, the training data includes the original data and associated segmented data. Labels for segmentation purposes include labels for each voxel in the segmented data. The segmented data may be generated and labeled using any method or process, for example, manually by an operator or automatically by one or more automatic methods. Different training data may be acquired for different segmentation tasks. For example, a first set of training data may be used to train a first network for segmenting brain data, while a second set of training data may be used to train a second network for segmenting heart data. The training data may be acquired at any point prior to inputting the training data into the trained network. The training data may include volumes of different resolutions or contrast. The training data may be updated after acquiring new data. The updated training data may be used to retrain or update the trained network.

The output of the GAN training process is a trained network 41 that is configured to input MR data and output segmented and classified MR data 33. For the segmentation and classification, single or multiple networks may be trained and used. One network may be trained to perform the segmentation task while a second network may be trained to perform the tissue classification.

The output of the trained network 41 for segmentation and tissue classification is input into a second trained network 43 that is configured for abnormality detection. The second trained network 43 is trained using deep learning techniques to input an image and identify abnormal regions. One method of identifying abnormal regions is by using a trained autoencoder network 43. An autoencoder is a neural network that is trained by unsupervised learning. The autoencoder is trained to learn reconstructions that are close to its original input. An autoencoder is composed of two parts, an encoder and a decoder. The encoder compresses the input data into a latent space. The decoder decompresses the latent space in order to attempt to reconstruct the input data. During training, the output of the decoder is compared to the original input to calculate a reconstruction error. Using multiple repetitions and adjustments, the autoencoder learns to minimize the reconstruction error. The output of the training process is a trained autoencoder network 43.

In application, autoencoder-based anomaly detection is a deviation-based anomaly detection method. The autoencoder may be trained adversarially to distinguish healthy case from pathological cases. The autoencoder 43 uses the reconstruction error on input data as an anomaly score. Data points with high reconstruction errors are considered to be anomalies. Only data with normal instances are used to train the autoencoder 43. After training, the autoencoder 43 will reconstruct normal data very well, while failing to do so with anomaly data, which the autoencoder 43 has not encountered. The output of the autoencoder 43 is anomaly data 35 that describes one or more regions in the first MR data that are unexpected. The abnormal regions provide guidance for further imaging protocols or sequences.

At act A240, an abbreviated acquisition protocol 45 is ordered as a function of the identified landmarks and identified abnormal regions. The abbreviated acquisition protocol 45 may include sequences that include scans of the identified abnormal regions. As a result of the scout analysis performed at acts A220-A230, the additional scans in the abbreviated acquisition protocol 45 may target regions of the organ where there might be abnormality, e.g. to generate additional focused high-resolution scans on the suspicious regions. The abbreviated acquisition protocol 45 may also include sequences or scans that are tailored to the patient and any specific issues. For example, if the suspected diagnosis is a tumor, the sequences and scans may be configured to acquire data relating to tumor diagnosis (in addition to the guidance provided by the analysis of the first MR data). In an example, the abbreviated acquisition protocol 45 may include one or more sequences such as structural (T1w, T2w, FLAIR) or diffusional (ADC, TraceW) scans. SWI or GRE may also be part of the abbreviated acquisition protocol 45. The abbreviated acquisition protocol 45 may include scans at a higher resolution than the scout protocol (the first MR data).

At act A250, second MR data 47 is acquired using the abbreviated acquisition protocol 45. The abbreviated acquisition protocol 45 may include multiple sequences. Each sequence may be performed and validated prior to moving on to the next sequence. Validation for the sequences of the abbreviated acquisition protocol 45 may include determining an alignment or misalignment of the second MR data 47 with respect to the matrices acquired from first MR data. For instance, using a simultaneous multi-slice protocol, slices from the second MR data 47 may be registered to the first MR data to ensure that misalignment is minimal. Additionally, or alternatively, the sequences of the abbreviated acquisition protocol 45 may be validated by checking that the quality of the acquisition is correct, e.g. there is an acceptable amount of motion, bias field, noise. Any automated technique may be used to check for the amount of motion, bias field, or noise. Thresholds may be defined for each of motion, bias field, or noise. If the threshold is exceeded, the sequence may fail validation. If a sequence or set of acquired MR data fails validation, the sequence may be repeated, altered to fix any issues and repeated, or skipped. The process and alteration may be performed automatically, or the procedure may pause and require input from a clinician. As mentioned above, the abbreviated acquisition protocol 45 may be stopped at any point after a sequence if the findings are not validated.

The second MR data 47 may include separate data for each sequence of the abbreviated acquisition protocol 45. For example, if there are five sequences in the abbreviated acquisition protocol 45, there may be five separate sets of MR data 47.

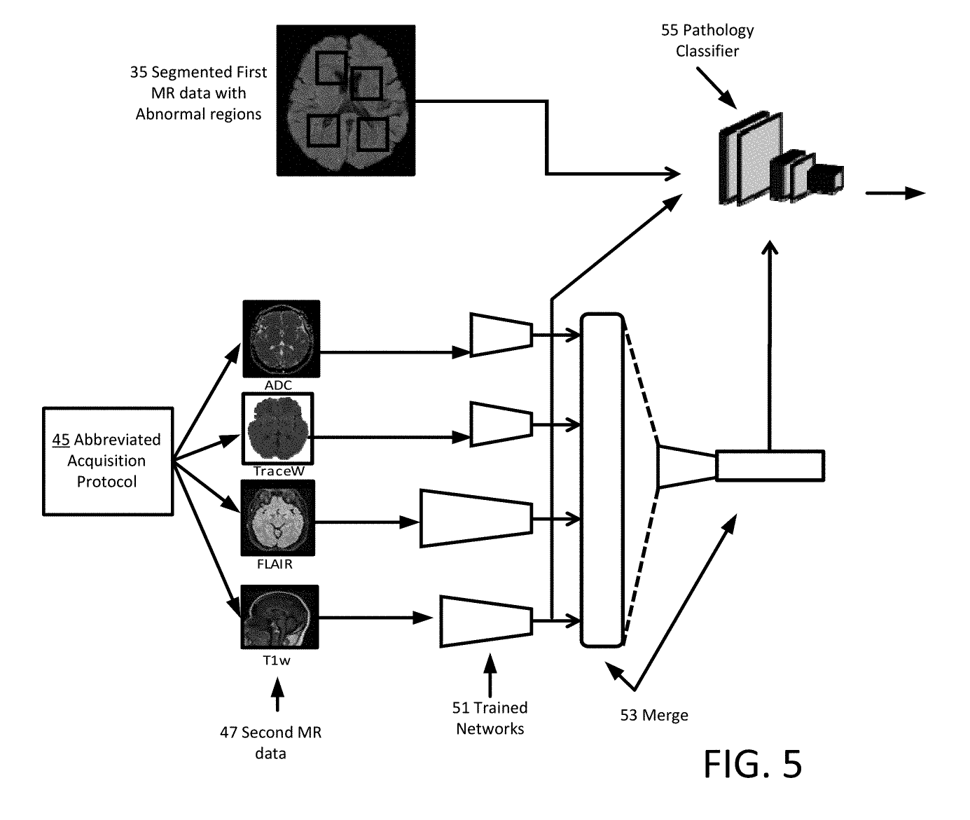

At act A260, anomalies are identified in the second MR data 47. Each portion or set or series of data acquired with the abbreviated acquisition protocol 45 is input into a separate trained network 51 configured to detect anomalies. FIG. 5 depicts a process of detecting anomalies. As depicted, the output data 47 of four separate sequences (ADC, TraceW, FLAIR, and T1w) of the abbreviated acquisition protocol 45 are input into four trained networks 51. Each network 51 may be trained offline to identify anomalies in the input data. Different deep learning or machine learning techniques may be used to train the networks 51. Each network 51 may include a different structure and may be trained separately from the other networks 51.

A DenseNet or other network arrangements may also be used for the trained networks 51 or other trained networks described above for segmentation or classification. A DenseNet connects each layer in a network to every other layer in a feed-forward fashion. For each layer in the DenseNet, the feature-maps of all preceding layers are used as inputs, and the output feature-map of that layer is used as input into all subsequent layers. In the DenseNet, for each layer, the feature maps of all preceding layers are used as inputs, and its own feature maps are used as inputs into all subsequent layers. To reduce the size of the network, the DenseNet may include transition layers. The layers include convolution followed by average pooling. The transition layers reduce height and width dimensions but leave the feature dimension the same. The neural network may further be configured as a U-net. The U-Net is an autoencoder in which the outputs from the encoder-half of the network are concatenated with the mirrored counterparts in the decoder-half of the network. Skip connections prevent the middle of the network from becoming a bottleneck.

Deep architectures include convolutional neural network (CNN) or deep belief nets (DBN), but other deep networks may be used. CNN learns feed-forward mapping functions while DBN learns a generative model of data. In addition, CNN uses shared weights for all local regions while DBN is a fully connected network (e.g., including different weights for all regions of an image). The training of CNN is entirely discriminative through back-propagation. DBN, on the other hand, employs the layer-wise unsupervised training (e.g., pre-training) followed by the discriminative refinement with back-propagation if necessary. In an embodiment, the arrangement of the trained network 51 is a fully convolutional network (FCN). Alternative network arrangements may be used, for example, a 3D Very Deep Convolutional Networks (3D-VGGNet). VGGNet stacks many layer blocks containing narrow convolutional layers followed by max pooling layers. A 3D Deep Residual Networks (3D-ResNet) architecture may be used. A Resnet uses residual blocks and skip connections to learn residual mapping.

Each of the trained networks 51 are defined as a plurality of sequential feature units or layers. Sequential is used to indicate the general flow of output feature values from one layer to input to a next layer. The information from the next layer is fed to a next layer, and so on until the final output. The layers may only feed forward or may be bi-directional, including some feedback to a previous layer. The nodes of each layer or unit may connect with all or only a sub-set of nodes of a previous and/or subsequent layer or unit. Skip connections may be used, such as a layer outputting to the sequentially next layer as well as other layers. Rather than pre-programming the features and trying to relate the features to attributes, the deep architecture is defined to learn the features at different levels of abstraction based on an input MR data with or without pre-processing. The features are learned to reconstruct lower level features (i.e., features at a more abstract or compressed level). For example, features for reconstructing an image are learned. For a next unit, features for reconstructing the features of the previous unit are learned, providing more abstraction. Each node of the unit represents a feature. Different units are provided for learning different features.

Various units or layers may be used, such as convolutional, pooling (e.g., max-pooling), deconvolutional, fully connected, or other types of layers. Within a unit or layer, any number of nodes is provided. For example, 100 nodes are provided. Later or subsequent units may have more, fewer, or the same number of nodes. In general, for convolution, subsequent units have more abstraction. For example, the first unit provides features from the image, such as one node or feature being a line found in the image. The next unit combines lines, so that one of the nodes is a corner. The next unit may combine features (e.g., the corner and length of lines) from a previous unit so that the node provides a shape indication. For transposed-convolution to reconstruct, the level of abstraction reverses. Each unit or layer reduces the level of abstraction or compression.

Each of the networks 51 may be trained using machine learning techniques to output a classification or probability of whether or not a region or feature is abnormal. Each of the networks 51 may also be configured to generate a deviation value for the region or feature. The output of the trained networks may be merged 53 to generate data relating to each region or feature in the scans. In an embodiment, the data may be limited to a determination of whether or not a region or feature is abnormal or not. The data may also include confidence data on the classification that is used in the merging process. Alternatively, the output data from the trained networks 51 may not be merged 53, but input separately into a pathology classifier 55 as described below.

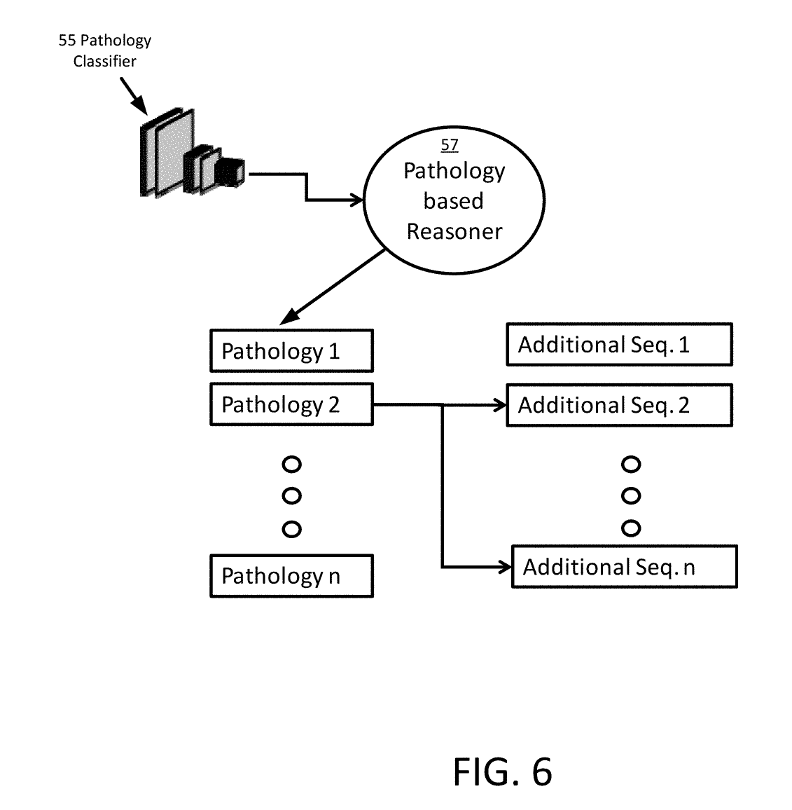

At act A270, one or more additional acquisition sequences are identified. FIG. 6 depicts a workflow for identifying additional sequences. The analysis of the abbreviated acquisition sequences and the scout acquisition sequence is used to determine if any additional sequences may be beneficial. If so, the additional sequences are configured and automatically ordered. The outputs and analysis of the previous scan may be input into a pathology classifier 55 that identifies a pathology. The pathology may be matched with the appropriate sequence using a pathology based reasoner 57 or other model. The additional sequence(s) may be ordered and performed. The pathology classifier 55 may be trained using ML or deep learning techniques to classify the results of the previous scans. In an embodiment, using unsupervised learning, a manifold space of deviations is created for automated clustering. Using actual pathological cases, clusters are matched to pathologies and therefore to additional suggested scans.

In another embodiment, supervised learning may be used with direct classification of the findings into pathologies and best next sequences. Findings are reported from each sequence of the abbreviated acquisition protocol 45 and therefore specific action(s) can be taken in accordance to the finding and sequence. Matching the pathology to the needed sequence may also be done by the pathology based reasoner 57 based on the findings from each sequence. After the addition acquisition sequences are determined, the one or more acquisition sequences are performed and the results may be displayed to an operator or stored for later use.

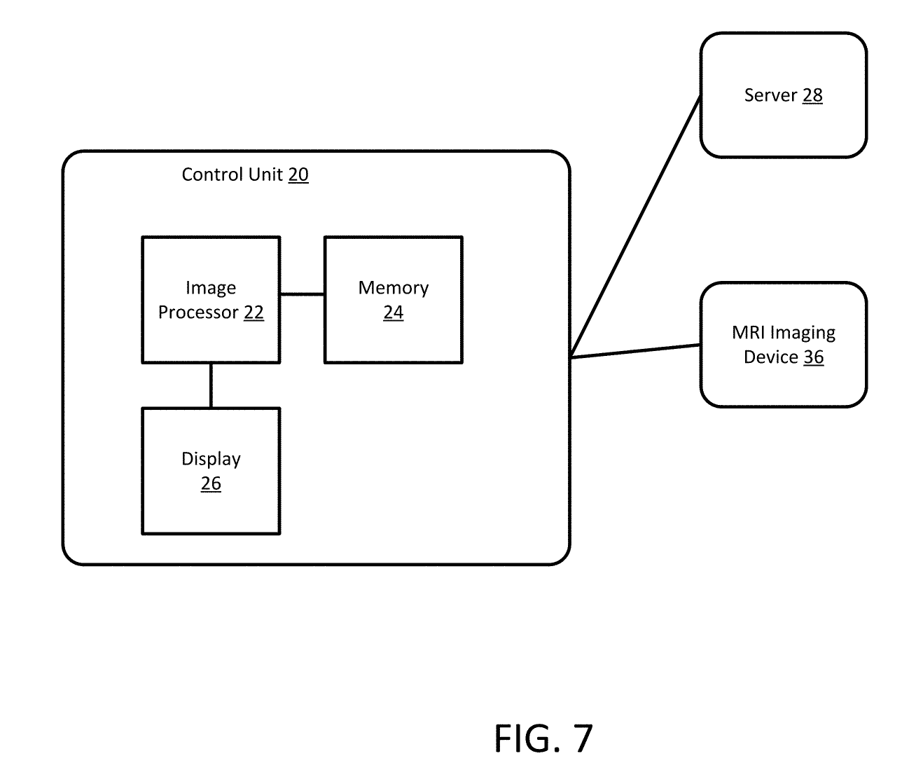

FIG. 7 depicts one embodiment of a control unit for automated acquisition of medical imaging data. The control unit includes an image processor 22, a memory 24, and a display 26. The control unit 20 may be connected with a server 28 and an MR imaging device 36. Additional, different, or fewer components may be provided. For example, network connections or interfaces may be provided, such as for networking between the control unit 20 and server 28. A workstation with a user interface may be provided for an operator to input data.

The MR imaging device 36 may be similar to the MR imaging device 36 as depicted in FIG. 1. The MR imaging device 36 is configured to acquire MR data that may be processed into one or more images or volumes by the control unit 20. The control unit 20 may provide commands to the MR imaging device 36. Alternatively, the MR imaging device 36 may function entirely on its own without any input from the control unit 20.

The image processor 22 (or processor) is a general processor, central processing unit, control processor, graphics processor, digital signal processor, three-dimensional rendering processor, image processor, application specific integrated circuit, field programmable gate array, digital circuit, analog circuit, combinations thereof, or other now known or later developed device for processing an image. The processor 22 is a single device or multiple devices operating in serial, parallel, or separately. The processor 22 may be a main processor of a computer, such as a laptop or desktop computer, or may be a processor for handling some tasks in a larger system, such as in the MR system. The processor 22 is configured by instructions, design, hardware, and/or software to perform the acts discussed herein.

The server 28 may be co-located with the control unit 20 or may be located remotely. The server 28 may connect to the MR system 100 or control unit 20 via a network. The network is a local area, wide area, enterprise, another network, or combinations thereof. In one embodiment, the network is, at least in part, the Internet. Using TCP/IP communications, the network provides for communication between the processor 24 and the server 28. Any format for communications may be used. In other embodiments, dedicated or direct communication is used.

The server 28 may include the processor 24 or group of processors. More than one server 28 or control unit 20 may be provided. The server 28 is configured by hardware and/or software. The processor 24 and/or server 28 are configured to perform the acts discussed above for automated acquisition workflow. The processor 24 and/or server 28 may access and implement the code stored in memory 24.

The memory 24 may be a graphics processing memory, a video random access memory, a random-access memory, system memory, cache memory, hard drive, optical media, magnetic media, flash drive, buffer, database, combinations thereof, or other now known or later developed memory device for storing data or video information. The memory 24 is part of the control unit 20, part of a database, part of another system, a picture archival memory, or a standalone device. The memory 24 may store MR data from the MR device 36.

The memory 24 includes an instruction set or computer code for implementing automated acquisition of medical imaging data. The memory 24 includes instructions for ordering a scout acquisition imaging sequence of a patient or organ localization. An abbreviated acquisition protocol 45 is automatically order, the abbreviated acquisition protocol 45 configured in part based on the scout acquisition. The abbreviated acquisition protocol 45 may include structural (T1w, T2w, FLAIR) and diffusional (ADC, TraceW) sequences at a minimum. Depending on the medical institution, SWI or GRE may also be part of the abbreviated acquisition protocol 45. In case of specific pathologies (Brain tumor), additional acquisitions, such as Perfusion, T1w with Contrast and more may be performed.

The memory 24 includes an instruction set or computer code to automate the abbreviated acquisition protocol 45 and then, from the results of the sequences, use deep learning trained networks to decide the appropriate next sequence(s). The instruction set may include three steps: scout images, abbreviated acquisition protocol 45 with minimal number of sequences, and additional sequences based on pathology findings.

The instructions for implementing the processes, methods and/or techniques discussed herein are provided on non-transitory computer-readable storage media or memories, such as a cache, buffer, RAM, removable media, hard drive, or other computer readable storage media. Non-transitory computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code, and the like, operating alone, or in combination. Likewise, processing strategies may include multiprocessing, multitasking, parallel processing, and the like.

The display 26 may be configured to display images to an operator. The display 26 may augment the images with additional information or overlays. The display 26 may be configured to display the images in two dimensions, three dimensions, or, for example, in augmented or virtual reality scenarios.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method for automated image acquisition of a patient using a magnetic resonance imaging system, the method comprising:
acquiring, by the magnetic resonance imaging system, first MR data using a first imaging protocol;
validating, by the processor, the first MR data;
ordering, by the processor, a second imaging protocol as a function of the validation of the first MR data;
acquiring, by the magnetic resonance imaging system, second MR data using the second imaging protocol;
validating, by the processor, the second MR data; and
ordering, by the processor, a third imaging protocol as a function of the validation of the first MR data and the second MR data.

2. The method of claim 1, wherein the first imaging protocol is performed at a lower resolution than the second imaging protocol.

3. The method of claim 1, wherein validating the first MR data comprises:
detecting, by the processor, landmarks in the first MR data using a network trained using deep reinforcement learning techniques;
scoring, by the processor, the landmark detection; and
validating, by the processor, the first MR data when the score exceeds a predefined threshold.

4. The method of claim 1, wherein the second imaging protocol comprises a plurality of sequences.

5. The method of claim 4, wherein each of the plurality of sequences are performed and validated prior to a subsequent sequence of the plurality of sequences is performed.

6. The method of claim 1, wherein validating the second MR data comprises:
identifying, by the processor, an acquisition matrix in the first MR data; and
checking, by the processor, that a geometry of the second MR data is correct the against acquisition matrix.

7. The method of claim 1, wherein validating the second MR data comprises:
validating an alignment of the second MR data.

8. The method of claim 1, wherein the first MR data is brain MR data and the second imaging protocol includes at least T1w, FLAIR, ADC, and TraceW sequences.

9. The method of claim 1, further comprising:
acquiring, by the magnetic resonance imaging system, third MR data using the third imaging protocol; and
displaying the third MR data.

10. A method for automated medical image acquisition of a patient, the method comprising:
acquiring, by a magnetic resonance imaging system, first MR data using a scout acquisition sequence;
detecting, by a processor, landmarks in the first MR data;
identifying, by the processor, abnormal regions in the first MR data;
determining, by the processor, an abbreviated acquisition protocol as a function of the identified landmarks and identified abnormal regions;
acquiring, by the magnetic resonance imaging system, second MR data using the abbreviated acquisition protocol;
identifying, by the processor, anomalies in the second MR data;
determining, by the processor, one or more additional acquisition sequences as a function of the identified anomalies; and
performing, by the magnetic resonance imaging system, the one or more additional acquisition sequences.

11. The method of claim 10, further comprising:
validating the scout acquisition sequence prior to determining the abbreviated acquisition protocol.

12. The method of claim 10, wherein identifying landmarks comprises:
identifying landmarks using a deep reinforcement trained agent.

13. The method of claim 10, wherein identifying abnormal regions comprises:
segmenting the first MR data using a network trained using an adversarial process;
inputting the segmented first MR data into a variable autoencoder network; and
identifying regions in the segmented first MR data with reconstruction errors higher than a predefined threshold as abnormal.

14. The method of claim 10, wherein anomalies in the second MR data are identified using a trained dense network.

15. The method of claim 10, wherein determining one or more additional acquisition sequences comprises:
identifying a pathological condition using a pathology classifier trained using machine learning to input one or more anomalies and output the pathological condition.

16. The method of claim 10, wherein the scout acquisition sequence comprises a 2 mm isotropic resolution sequence.

17. The method of claim 10, wherein the abbreviated acquisition protocol comprises a plurality of sequences.

18. A system for automated medical image acquisition of a patient, the system comprising:
a magnetic resonance imaging device configured to acquire first MR data using a first imaging protocol and second MR data using a second imaging protocol;
a memory configured to store the first MR data and second MR data; and
a control unit configured to validate the first MR data, order the second imaging protocol as a function of the validation of the first MR data, validate the second MR data, and order a third imaging protocol as a function of the validation of the first MR data and second MR data.

19. The system of claim 18, wherein the control unit is further configured to detect anomalies in the first MR data and generate the second image protocol as a function of the detected anomalies.

20. The system of claim 18, further comprising:
a display configured to display the first MR data and second MR data.
